(19) United States
(12) Patent Application Publication
(10) Pub. No.: US 2014/0255459 A1
(43) Pub. Date: Sep. 11, 2014

(54) METHOD OF SURFACE TREATMENT OF MICRO/NANOPARTICLES BY CHEMICAL MEANS AND ITS APPLICATION TO OBTAINING A PIGMENT COMPOSITION INTENDED FOR THE FIELD OF COSMETICS, PAINT OR INKS

(71) Applicant: SIKEMIA, Clapiers (FR)

(72) Inventors: Franck Martin, Montpellier (FR); Johanne Moineau, Chartres (FR); Aude Mezy, Jacou (FR); Guillaume Gracy, Montpellier (FR)

(21) Appl. No.: 14/349,963

(22) PCT Filed: Oct. 5, 2012
(86) PCT No.: PCT/EP2012/069772

§ 371 Date: Apr. 4, 2014

(30) Foreign Application Priority Data
Oct. 5, 2011 (FR) 1159001

(51) Int. Cl. C09C 3/08; A61Q 1/06; A61Q 1/10; A61K 8/55; A61Q 1/02

(52) U.S. Cl. CPC C09C 3/08; A61K 8/55; A61Q 1/02; A61Q 1/10; A61Q 1/06
USPC 424/401; 424/63; 424/64; 556/19; 106/402; 106/417; 106/418; 556/173

(57) ABSTRACT

The subject matter of the invention is a method of surface modification of a pigment or of a composite pigment comprising at least a metal oxide, a metal complex or a derivative thereof, and the use of these pigments or composite pigments thus obtained for cosmetics, paint or inks.

The subject matter of the invention is a method of surface modification of a pigment or a composite pigment comprising at least a metal oxide, a metal complex or a derivative thereof, and the use of these pigments or composite pigments thus obtained for cosmetics, paint or inks.

Inorganic pigments can be used for their colouring power in cosmetic products, but also in paint and in inks. It is required of these pigments to be able to disperse in a homogeneous manner with the desired colour rendition, while at the same time exhibiting good stability when they are formulated for these different application fields.

Unfortunately, inorganic pigments such as metal oxides or inorganic composite pigments comprising them tend naturally to aggregate on account of their size, their high density and their natural affinity, particularly when it is attempted to disperse them in “eco-friendly” (non-toxic) solvents.

In addition, these pigments also have a tendency to aggregate when they are conserved in powder form before their formulation and their final use, which makes their storage and their conservation in crude form difficult after their production.

It thus remains desirable to be able to improve the dispersion and the stability of inorganic pigments, whether in solvents compatible with their cosmetic use, such as aqueous solvents, or in other solvents compatible with their final destination, but also to be able to improve their stability when they are conserved in dry form with good dispersion capacity in the solvent that will be used in the final formulation.

In addition and in order to homogenise their production, it would be desirable to be able to have available a method of producing these pigments, making them able to be formulated differently according to their final destination (cosmetics, paint or ink for example) with good capacities of dispersion and stability.

This is precisely the subject matter of the present invention.

The aim of the present invention is thus to be able to provide a pigment of inorganic nature with dispersive capacity and high stability, dispersed in a solvent or in dry form, and obtained by a simple and easy to implement method, such a pigment being able to be particularly formulated with solvents compatible with its cosmetic use.

The inventors have highlighted a novel method of surface modification of a substrate, particularly of a pigment intended to confer on said substrate interesting properties, particularly in the cosmetics field. This method consists in grafting, in one or more steps, on the surface of the substrate, groups providing a new surface functionality to the substrate and thus modifying its properties. This method is characterised by grafting by chemical means via a coupling agent. The invention also relates to the substrate obtained by this method as well as the diverse applications of the functionalised substrate.

During this method of grafting, the coupling agent is fixed on the substrate (grafting) by reaction of one of its functions with the surface of said substrate thanks to the formation of a stable covalent or iono-covalent bond.

The inventors have demonstrated that such a surface modification of said substrate by this method, in particular when it is applied to an inorganic pigment or to a composite pigment, makes it possible to obtain a certain number of advantages:
Grafting by chemisorption brings into play important bond energies and does so via an irreversible reaction. The formation of covalent or iono-covalent bonds thus assures the grafting has great stability over time;
The method makes it possible to treat by the same coupling agent a large variety of substrates (metals, alloys, metal oxides, etc.);
This method enables the deposition of coupling agent in the form of monolayers, thus minimising the quantity of coupling agent necessary and avoiding the masking of the terminal functions. In fact, the efficiency of the properties conferred on the substrate depend on the accessibility of the surface terminal functions;
The method proves to be, once developed, simple to execute and inexpensive;
The grafting method is implemented in easier conditions than that of the prior art, for example under ambient, non-anhydrous conditions, which makes it possible to avoid the use of organic solvents.

Thus, the invention relates to a method of modifying substrates (inorganic pigments, composite pigments) aiming to confer thereon interesting properties in the field of cosmetics, paint or inks. The method consists in grafting, in one or more steps on the surface of the substrate, groups having properties enabling applications in the sector of cosmetics, paint or inks.

The coupling agent is grafted onto the surface of the substrate by immersion of the substrate in a solution containing the coupling agent.

The functionalised substrate is then extracted from the grafting solution then dried.

The subject matter of the present invention is a method of surface modification of a pigment or a composition comprising at least a pigment, an excipient and, if need be, a lake, said pigment being a metal oxide, a metal complex or a derivative thereof, characterised in that it comprises the following steps:

A) placing in contact said pigment or said composition comprising at least a pigment with a coupling reagent in a solvent and in conditions making it possible to form a chemical bond, preferably covalent or iono-covalent, between said pigment and said coupling reagent; and

B) recovering said pigment or said composition comprising at least a pigment, the surface of which has thus been modified.

Coupling agent or reagent is here taken to designate any molecule comprising at each end a chemical function (reagent known as bi-functional). In general, a carbon chain plays the role of spacer between the 2 functions. One of these functions is capable in suitable conditions of reacting with said substrate and thus enables the attachment of said coupling agent to the surface of the substrate. Once this coupling agent is fixed on the surface of the substrate by its first reactive function, the second chemical function, designated herein terminal function, is going to provide the new property on the surface of the substrate thus chemically modified.

Thus, under a particular aspect, the method of surface modification according to the present invention is characterised in that said pigment or said composition comprising at least a pigment is intended to be used in cosmetics, in inks or in paint, preferably in cosmetics.

In a preferred embodiment, the method of surface modification according to the invention is characterised in that said coupling reagent further comprises a function that does not react with the surface of said pigment, but which provides the desired physical-chemical property.

Among these desired physical-chemical properties may be cited for example, but without being limited thereto, hydrophobic, hydrophilic, lipophilic, lubricant, bactericide, repulsive, attractive, colorant, luminescent characteristics or the immobilisation of biomolecules provided by different chemical functions which may be for example carboxylic acid, amine, thiol, epoxide functions and derivatives thereof. These sought after characteristics can also come from the carbon chain on account, for example, of the number of carbons or the presence of aromatic cycles and finally, they may be due to the presence of different chemical elements in ionic form.

Also in an embodiment, said pigment is a particle, porous or not, being able to exist in different geometric shapes such as for example, but without being limited thereto, a sphere, a rod, a wire, a tube, the size or diameter of which may be comprised between 5 μm and 125 nm, preferably between 5 μm and 100 nm, between 7.5 μm and 75 nm, between 10 μm and 75 nm or between 500 μM and 1 nm.

Among the pigments which may be implemented in the present invention, pigments may be cited selected from the groups of pigments consisting of:

1) Inorganic pigments; and

2) Composite pigments.

The subject matter of the present invention is a method of surface modification according to the present invention, characterised in that said excipient is selected from excipients of natural or synthetic origin, preferably selected from the following group:
Mica of formula X₂Y₄₋₆Z₈O₂₀(OH,F)₄ with X═K, Na, Ca, Ba, Rb, Cs; Y═Al, Mg, Mn, Cr, Ti, Li etc. and Z═Si, Al, Fe³⁺, Ti,
Synthetic fluorphlogopite of formula KMg₃(AlSI₃O₁₀)F₂,
Talc of formula Mg₃Si₄O₁₀(OH)₂,
Sericite of formula KAl₂ [(OH,F)₂|AlSi₃O₁₀]/KAl₂ [(OH,F)₂|AlSi₃O₁₀];

Silica of formula SiO₂; and

Alumina of formula Al₂O₃.

In a preferred manner, in the method of surface modification according to the invention, said pigment is an inorganic pigment being able to be composed of the following different chemical elements: F, Na, Mg, Al, Si, P, S, K, Ca, Ti, V, Cr, Mn, Fe, Co, Ni, Cu, Zn, Se, Zr, Mo, Ru, Cd, Sn, Sb, W.

Preferably, said pigment is an inorganic pigment selected from the group constituted of the following pigments, Black Iron Oxide, Red Iron Oxide, Yellow Iron Oxide, Manganese Violet, Ultramarine Blue, Titanium Dioxide, Chromium Oxide Green, Zinc Oxide, Iron Blue, Chromium Oxide Hydrated.

Under another aspect, said pigment is a composite pigment formed of one or more inorganic pigments associated with one or more excipients in the presence or not of a lake.

Under a particular aspect, the subject matter of the invention is a method of surface modification according to the invention, characterised in that said pigment is a composite pigment comprising one or more inorganic pigments, an excipient and, if need be, a lake selected from the group constituted of the following composite pigments:
Mica (59 to 69%), TiO₂ (18 to 28%), Fe₃O₄ (1 to 6%), FeO(OH) (1 to 2%) and Red7Calcium (8 to 12%);
Mica (56 to 66%), TiO₂ (10 to 20%), Fe₃O₄ (2 to 12%), FeO(OH) (1 to 11%) and Red7Calcium (8 to 12%);
Synthetic fluorphlogopite (18 to 28%), TiO₂ (54 to 64%) and Red40Aluminium (13 to 23%);
Synthetic fluorphlogopite (23 to 33%), TiO₂ (57 to 67%) and Red7Calcium (5 to 15%);
Mica (48 to 58%), TiO₂(32 to 42%) and Red7Calcium (8 to 12%);
Mica (46 to 56%), TiO₂ (34 to 44%) and Red7Calcium (8 to 12%);
Mica (50 to 60%), TiO₂ (29 to 39%) and Red7Calcium (10 to 14%);
Mica (49 to 59%), TiO₂ (36 to 46%) and Red7Calcium (3 to 7%);
Mica (46 to 56%), TiO₂ (34 to 44%) and Red28Aluminium (5 to 15%);
Mica (44 to 54%), TiO₂ (32 to 42%) and Blue1Aluminium (9 to 19%);
Mica (48 to 58%), TiO₂ (28 to 38%) and Yellow5Aluminium (9 to 19%);
Mica (48 to 58%), TiO₂ (28 to 38%) and Red6Sodium (9 to 19%);
Mica (47 to 57%), TiO₂ (31 to 41%) and Red30Aluminium (7 to 17%).
Mica (43 to 54%) and TiO₂ (46 to 57%);
Mica (55 to 65%) and TiO₂ (45 to 55%);
Mica (62 to 73%) and TiO₂ (27 to 38%);
Mica (77 to 87%) and TiO₂ (13 to 23%);
Mica (64 to 74%) and TiO₂ (26 to 36%);
Mica (52 to 63%) and TiO₂ (37 to 48%);
Mica (47 to 58%) and TiO₂ (42 to 53%);
Mica (45 to 56%) and TiO₂ (44 to 55%);
Mica (43 to 54%) and TiO₂ (46 to 57%);
Mica (37 to 48%) and TiO₂ (52 to 63%);
Mica (54 to 64%) and Fe₂O₃ (36 to 46%);
Mica (52 to 62% and Fe₂O₃ (38 to 48%);
Mica (62 to 72%) and Fe₂O₃ (28 to 38%); and
Mica (55 to 70%), TiO₂ (28 to 38%) and Fe₂O₃ (2 to 7%).

In the surface modification methods according to the invention, said solvent is characterised in that it is selected from “eco-friendly” (non-toxic) or organic solvents.

When the method of the invention is intended to modify the surface of said pigments for a cosmetic use of these pigments thus modified, it is preferred to use “eco-friendly” (non-toxic) solvents.

Among “eco-friendly” (non-toxic) solvents, in particular but without being restricted thereto cosmetically acceptable solvents are preferred, even more preferably selected from the group constituted of the following “eco-friendly” (non-toxic) solvents: ethanol, water, ethanol/water mixture in proportions ranging from 100%/0% to 0%/100%.

Among organic solvents, those selected from the group constituted of the following organic solvents are preferred: tetrahydrofuran, dimethylsulphoxide, dimethylformamide, cyclohexane, pentane, acetone, toluene, dichloromethane, isopropanol.

Under another preferred aspect, said coupling reagent is selected from the group constituted of coupling reagents of generic formula:

Y-E-Z (I)

in which:

Y (function providing the desired physical-chemical property or sought after characteristic) is a known organic function, a colorant, luminescent, pH-dependent, thermo-sensitive chemical group or a biomolecule;

E (spacer) is a radical selected from alkylene, alkenyl, alkynyl, aryl radicals, preferably C2 to C60 for the carbon chain of alkylene, alkenyl and alkynyl radicals or a poly(oxyethylene) radical comprising a number of oxyethylene units comprised between 4 and 500, preferably less than 100 and less than 50;

Z (grafting function on the pigment) is a radical selected from: —PO(OX)₂ (with X═H, Me, and, iPr, tBu, Na, L₁, NH₄⁺, K), —O—PO(OX)₂ (with X═H, Me, and, iPr, tBu, Na, L₁, NH₄⁺, K), —COOH, —SH, —SO₃H, quaternary ammonium salts.

In a particularly preferred embodiment, said coupling reagent is selected from the group constituted of the following coupling reagents:
Phosphonic acids and derivatives thereof such as octylphosphonic acid, dodecylphosphonic acid, octadecylphosphonic acid, (2-{2-[2-(2-hydroxy-ethoxy)-ethoxy]-ethoxy}-ethyl) phosphonic acid, sodium diphosphonate;
Carboxylic acids of C3 to C60 and derivatives thereof such as octanoic acid, dodecanoic acid, stearic acid, oleic acid, linoleic acid;
Thiols and derivatives thereof such as octadecanethiol; and
Amine functions and derivatives thereof such as octadecylammonium.

According to an also preferred embodiment, the method of surface modification according to the present invention is characterised in that it comprises the following steps:

a) placing in contact said pigment or said composition comprising at least an inorganic pigment with a coupling reagent in a solvent and in conditions making it possible to form a chemical bond, preferably covalent or iono-covalent, between said pigment and said coupling reagent;

b) eliminating the coupling reagent not having reacted with said pigment;

c) if need be, washing the pigment thus modified;

d) if need be, drying the pigment thus modified at its surface obtained at step b) or c) or the pigment composition; and

e) recovering said pigment or said composition comprising at least an inorganic pigment, the surface of which has thus been modified.

According to an also preferred embodiment, the method of surface modification according to the present invention is characterised in that it comprises the following steps:

a) placing in contact said pigment or said composition comprising at least a pigment with a coupling reagent in a solvent and in conditions making it possible to form a covalent or iono-covalent bond between said pigment and said coupling reagent;

b) eliminating the coupling reagent not having reacted with said pigment;

c) if need be, washing the pigment thus modified;

d) drying the pigment thus modified on its surface obtained at step b) or c) or the pigment composition; and

e) if need be, recovering said pigment or said composition comprising at least an inorganic pigment, the surface of which has thus been modified, in the form of a powder.

Also preferably, the steps of washing (and/or rinsing) and recovering the pigment, the surface of which has been modified, are carried out in the presence of solvent by mechanical separation (decantation, centrifugation), filtration in depth or on support preferably by filtration on membrane support.

Also preferably, the drying step is carried out by heating, preferably at a temperature comprised between 20° C. and 150° C., preferably 40° C. to 80° C.

Also preferably, the steps of washing and/or rinsing are followed by a step in which the pigment or the pigment composition obtained is subjected to ultrasounds.

Under another aspect, the subject matter of the present invention is a method of preparing a pigment powder or a composition comprising at least an inorganic pigment, preferably intended to be used in cosmetics, in paint or in inks, preferably in cosmetics, characterised in that it comprises the following steps:

I) modifying the surface of the pigment by the method according to the present invention; and

II) a step of recovering said pigment or said composition comprising at least a pigment, the surface of which has thus been modified obtained at step 1), in the form of a powder.

Under yet another aspect, the subject matter of the present invention is a method of preparing a pigment powder or a dry composition comprising at least a pigment, said pigment being selected from inorganic, composite or organic pigments, preferably said powder or dry composition being intended to be used in cosmetics, in paint or in inks, preferably in cosmetics, characterised in that it comprises the following steps:

1) modifying the surface of the pigment by the method according to the present invention and in which method a pigment or a composition comprising at least a pigment is placed in contact with a reagent or coupling agent in a solvent and in conditions making it possible to form a chemical bond, preferably covalent or iono-covalent, between said pigment and said coupling reagent, and characterised in that the solvent/coupling reagent mixture represents by weight less than 50% of the weight of pigment, preferably, less than 25%, less than 10%, a value comprised between 3% and 10% being most preferred;

2) after mixing, a step of drying, preferably by heating, the mixture obtained at step 1) so as to obtain a pigment powder or a dry composition, preferably by heating to a temperature comprised between 20° C. and 150° C., even more preferably between 20° C. and 80° C., in a more preferred manner between 40° C. and 80° C. Preferably, the mixer is equipped with a thermoregulator which makes it possible to regulate the temperature during the drying step.

3) If need be, a step of recovering said pigment powder or said composition in the form of a powder.

The drying temperature and time are such that they are sufficient to evaporate the solvent and obtain a powder or dry composition, for example 12 hours at 80° C. or 24 hours at 40° C., but they can vary according to the nature of the solvent and/or the coupling reagent used.

Such a method in which the mass ratio between the mixture [solvent/coupling agent] and pigment represents less than 50%, preferably, less than 25%, less than 10%, a value comprised between 3% and 10% being more preferred, has the advantage of obtaining a more rapid drying and/or at a drying temperature below that normally used. This makes it possible particularly to be able to apply such a method to pigments of organic type that are more fragile and liable to be denatured than inorganic pigments. In addition, such a method makes it possible to avoid any complementary step of washing, decantation, filtration or other step intended to eliminate the solvent and/or free reagent not having reacted with the surface of the pigment.

The principle of this method known as method by dry means is grafting by placing in contact the coupling agent and pigments (substrates). The pigments thus treated may be organic or inorganic (or composites). This method also makes it possible to treat excipients and effect pigments.

The organic pigments may be particularly selected from the list identified in the following table 9 (see example 7)

In a preferred embodiment, the solvent containing the reagent or coupling agent is vaporised on the surface of the pigment (substrate) or composition to be treated, then if need by mixing in a homogeneous manner, particularly by means of a mixer or stirrer before the drying step.

The weight percent of the coupling agent in solution varies as a function of the weight ratio of the couple (coupling agent/pigment to be treated). It is generally considered as sufficient from 3%. The choice of the solvent (H₂O, ethanol (EtOH) or H₂O/EtOH mixture) also varies as a function of the coupling agent/pigment couple. Conventional organic solvents may also be employed, particularly of hexane, heptane, cyclohexane, benzene, xylene or toluene type, but current environmental considerations and those linked to health (particularly in cosmetic use) tend to see their uses reduced.

Under another aspect, the subject matter of the present invention is a pigment powder coated with a monolayer of coupling agent in which the free terminal functions (Y) confer to the pigments the desired physical-chemical properties, said pigment powder being capable of being obtained by a method according to the present invention.

According to yet another aspect, the powder of dispersed pigments is capable of being obtained by a method of modification according to the invention and in which method the surface modification is intended to provide properties of dispersion, stability (time, temperature, radiation), texturing but can also make it possible to simplify the formulation, to protect the pigment or instead to limit the use of toxic products.

Preferably, said pigment or pigment composition capable of being obtained by a method of surface modification according to the invention, intended to be used in cosmetics, in paint or in inks, preferably in cosmetics, is characterised in that it is contained in an eye shadow, a blush, a lipstick, a cream, a gel, a soap bar or any form of cosmetic product.

According to another aspect, the subject matter of the present invention is a method of improving the dispersion of pigments, characterised in that it implements the steps of the method of surface modification according to the present invention.

The subject matter of the invention is also a pigment or pigment composition capable of being obtained by a method of surface modification according to the invention, characterised in that the method implements the following elements:

1)—Coupling reagent: Phosphonic acid (C18);
Pigments:
Inorganic pigments selected from: Black Iron Oxide; Red Iron Oxide; Yellow Iron Oxide; Titanium Dioxide; Chromium Oxide Green; Ultramarine Blue; Zinc Oxide; Iron Blue; or Chromium Oxide Hydrated;
Composite pigments;
   Excipients: Mica; Fluorphlogopite; or Talc,

or

2)—Coupling reagent: Carboxylic acid (C18)
Pigments:
Inorganic pigments selected from: Black Iron Oxide; Red Iron Oxide; Yellow Iron Oxide; Titanium Dioxide; Chromium Oxide Green; Zinc Oxide; or Chromium Oxide Hydrated;
Composite pigments selected from: Mica (59 to 69%), TiO₂ (18 to 28%), Fe₃O₄ (1 to 6%), FeO(OH) (1 to 2%) and Red7Calcium (8 to 12%); Mica (56 to 66%), TiO₂ (10 to 20%), Fe₃O₄ (2 to 12%), FeO(OH) (1 to 11%) and Red7Calcium (8 to 12%); or Mica (46 to 56%), TiO₂ (34 to 44%) and Red28Aluminium (5 to 15%);
   Excipient: Talc,

or

3)—Coupling reagent: Thiol (C18)
Pigments:
Inorganic pigments selected from: Black Iron Oxide; Red Iron Oxide; Yellow Iron Oxide; Titanium Dioxide; Chromium Oxide Green; Zinc Oxide; or Iron Blue;
Composite pigments selected from: Mica (59 to 69%), TiO₂ (18 to 28%), Fe₃O₄ (1 to 6%), FeO(OH) (1 to 2%) and Red7Calcium (8 to 12%); or Mica (56 to 66%), TiO₂ (10 to 20%), Fe₃O₄ (2 to 12%), FeO(OH) (1 to 11%) and Red7Calcium (8 to 12%),

or

4)—Coupling reagent: Ammonium salts (C18)
Pigments:
Inorganic pigments selected from: Manganese Violet; or Zinc Oxide;
Composite pigments;
   Excipients: Mica; Fluorphlogopite; Talc; Silica; Sericite; or Alumina,

or

5)—Coupling reagent: (2-{2-[2-(2-hydroxy-ethoxy)-ethoxy]-ethoxy}-ethyl) phosphonic acid
Pigments:
Inorganic pigments selected from: Black Iron Oxide; Red Iron Oxide; Yellow Iron Oxide; Ultramarine; or Zinc Oxide;
Composite pigments: Mica (59 to 69%) TiO₂ (18 to 28%) Fe₃O₄ (1 to 6%) FeO(OH) (1 to 2%) Red7Calcium (8 to 12%);

Under a final aspect, the invention relates to an eye shadow formulation of the following composition:
Mica, Lauroyl Lisine 48% by weight;
Pigments modified by a method according to the invention: 40% by weight;
Petrolatum 2% by weight;
Dimethicone 5% by weight; and
Caprilic/Capric Triglyceride 5% by weight;

The invention also relates to a foundation formulation of following composition:
Stearoyl inulin 2% by weight;
Isotridecyl isononanate 5% by weight;
Dimethicone copolyol 1.7% by weight;
Cyclomethicone 13.4% by weight;
Pigments modified by a method according to the invention: 6.9% by weight;
Sodium chloride 1% by weight;
Methylparaben 0.2% by weight; and
Purified water qs.

Other characteristics, aspects and advantages of the present invention will become clearer on reading the detailed description that follows and by referring to the figures.

### EXAMPLE 1

### Fixation of the Coupling Agent on the Substrate by Reaction of One Of its Functions with the Surface Thanks to the Formation of a Stable Covalent or Iono-Covalent Bond

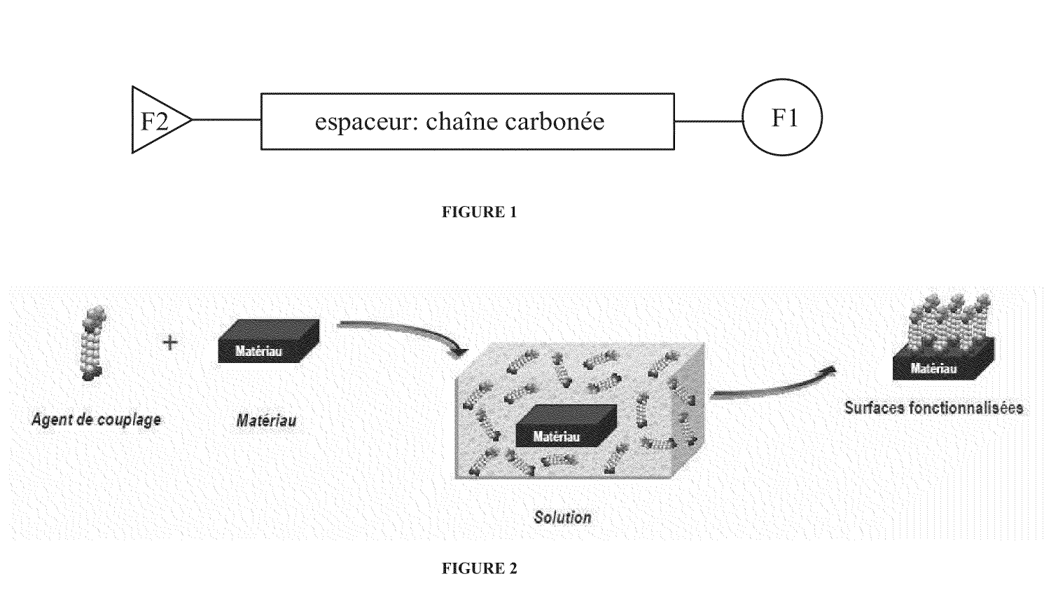

### (See FIG. 1)

The coupling agent is grafted onto the surface of the substrate by immersion of the substrate in a solution containing the coupling agent.

### (See FIG. 2)

The functionalised substrate is then extracted from the grafting solution then dried.

Protocol

The graftings take place in three steps:
Grafting reaction by chemical means between the coupling agent solubilised in a suitable solvent and the surface of the materials (pigments) to be modified,
Elimination of the grafting solution and washing of the modified material,
Drying the functionalised material (pigment).

### A) Step 1:

The grafting step is carried out by chemical reaction between a coupling agent at desired concentration and the surface of a pigment to be modified, in the selected solvent (EtOH, H₂O or H₂O/EtOH mixture) for a desired time.

Example of Method Implemented for Step 1:
A solution of coupling agent is prepared by dissolution of the coupling agent in 10 ml of solvent.

The concentration of the solution varies between 10⁻¹ and 10⁻³ mol.l⁻¹ as a function of the couple (selected coupling agent/substrate to be treated). The choice of the solvent (H₂O, EtOH or H₂O/EtOH mixture) also varies as a function of the coupling agent/substrate couple. Conventional organic solvents may also be employed but current environmental considerations tend to see their uses reduced.
0.5 g of pigments is placed (substrate) in a pill machine to which is added 10 ml of solution of coupling agent.

Dynamic Condition: The solution obtained is placed under magnetic stirring for a time t at ambient temperature (10-35° C.).

Static Condition: The chemical reaction takes place without mechanical stirring at ambient temperature (10-35° C.), for a time t.

The time t varies between 5 minutes and 72 h as a function of the coupling agent/substrate couple.

### B) Step 2:

The grafting solution is eliminated by filtration then the material (modified substrate) is washed in order to assure the complete elimination of all the physisorbed elements.

Example of Method Implemented for Step 2
The solution contained in the pill machine is decanted into a centrifuge tube then made up to ¾ with EtOH.
After a centrifugation for 10 minutes at a speed of 20,000 rpm, the filtrate is eliminated from the centrifuge tube.
The centrifuge tube is again made up to ¾ with EtOH and is placed in an ultrasound tank for 10 minutes.
The solution is then centrifuged according to the same conditions.

### C) Step 3:

Several steps of drying the modified material are then carried out.

Example of Method Implemented for Step 3

After elimination of the filtrate, the residual solvent is evaporated under vacuum (5.10⁻¹ mbar) for 15 minutes.
The powders of functionalised pigments (substrates) are decanted into a glass pill machine and dried for 12 hours.

The temperature of the oven varies between 20 and 150° C. as a function of the tolerance of the different substrates to the thermal conditions.

Thereafter, characterisation tests specific to the sought after properties are carried out in order to determine whether the surface of the substrates has been suitably grafted.

### EXAMPLE 2

### Inorganic Pigments (Substrates)

The substrate to be treated may be a pigment of mineral origin, a metal oxide or a metal complex usually employed in cosmetics, in paint or in inks. These are solids, porous or not, which exist in the form of powder, a non-exhaustive list of which is presented below:

TABLE 1

| Inorganic pigment | Chemical formula |
|---|---|
| Black Iron Oxide | Fe₃O₄ |
| Red Iron Oxide | Fe₂O₃ |
| Yellow Iron Oxide | Fe₂O₃•H₂O |
| Manganese Violet | H₄MnNO₇P₂ |
| Ultramarine Blue | Na₆Al₄Si₆S₄O₂₀ |
| Titanium dioxide | TiO₂ |
| Chromium Oxide Green | Cr₂O₃ |
| Zinc Oxide | ZnO |
| Iron Blue | NH₄•Fe•Fe(CN)₆ |
| Chromium Oxide Hydrated | Cr₂O_{3,} H₂O |

### EXAMPLE 3

### Excipients

This is a chemical substance that may be of natural or synthetic origin, which is other than the active ingredient and which does not interfere therewith. Its role is to give consistency to the product or different physical characteristics.

TABLE 2

| Excipient | Chemical formula |
|---|---|
| Mica | X₂Y₄₋₆Z₈O₂₀(OH,F)₄ with X = K, Na, |
| | Ca, Ba, Rb, Cs; Y = Al, Mg, Mn, Cr, |
| | Ti, Li etc. and Z = Si, Al, Fe³⁺, Ti |
| Fluorphlogopite | KMg₃(AlSI₃O₁₀)F₂ |
| Talc | Mg₃Si₄O₁₀(OH)₂ |
| Sericite | KAl₂[(OH,F)₂\|AlSi₃O₁₀]/ |
| | KAl₂[(OH,F)₂\|AlSi₃O₁₀] |
| Silica | SiO₂ |
| Alumina | Al₂O₃ |

### EXAMPLE 4

### Composite Pigments (Substrates)

Association of at least an inorganic pigment based on excipient and, if need be, a lake.

For the excipients (see table 2):
For the lakes (see table 3):

TABLE 3

| Inorganic pigments + Excipients | Lake |
|---|---|
| Mica (59 to 69%) TiO₂ (18 to 28%) | Red7Calcium (8 to 12%) |
| Fe₃O₄ (1 to 6%) FeO(OH) (1 to 2%) | |
| Mica (56 to 66%) TiO₂ (10 to 20%) | Red7Calcium (8 to 12%) |
| Fe₃O₄ (2 to 12%) FeO(OH) (1 to 11%) | |
| Fluorphlogopite (18 to 28%) TiO₂ | Red40Aluminium (13 to 23%) |
| (54 to 64%) | |
| Fluorphlogopite (23 to 33%) TiO₂ | Red7Calcium (5 to 15%) |
| (57 to 67%) | |
| Mica (48 to 58%) TiO₂(32 to 42%) | Red7Calcium (8 to 12%) |
| Mica (46 to 56%) TiO₂ (34 to 44%) | Red7CaIcium (8 to 12%) |
| Mica (50 to 60%) TiO₂ (29 to 39%) | Red7Calcium (10 to 14%) |
| Mica (49 to 59%) TiO₂ (36 to 46%) | Red7Calcium (3 to 7%) |
| Mica (46 to 56%) TiO₂ (34 to 44%) | Red28Aluminium (5 to 15%) |
| Mica (44 to 54%) TiO₂ (32 to 42%) | Blue1Aluminium (9 to 19%) |
| Mica (48 to 58%) TiO₂ (28 to 38%) | Yellow5Aluminium (9 to 19%) |
| Mica (48 to 58%) TiO₂ (28 to 38%) | Red6Sodium (9 to 19%) |
| Mica (47 to 57%) TiO₂ (31 to 41%) | Red30Aluminium (7 to 17%) |

TABLE 4
Inorganic pigments + Excipients

Mica (43 to 54%) TiO₂ (46 to 57%)
Mica (55 to 65%) TiO₂ (45 to 55%)
Mica (62 to 73%) TiO₂ (27 to 38%)
Mica (77 to 87%) TiO₂ (13 to 23%)
Mica (64 to 74%) TiO₂ (26 to 36%)
Mica (52 to 63%) TiO₂ (37 to 48%)
Mica (47 to 58%) TiO₂ (42 to 53%)
Mica (45 to 56%) TiO₂ (44 to 55%)
Mica (43 to 54%) TiO₂ (46 to 57%)
Mica (37 to 48%) TiO₂ (52 to 63%)
Mica (54 to 64%) Fe₂O₃ (36 to 46%)
Mica (52 to 62%) Fe₂O₃ (38 to 48%)
Mica (62 to 72%) Fe₂O₃ (28 to 38%)
Mica (55 to 70%) TiO₂ (28 to 38%) Fe₂O₃ (2 to 7%)

### EXAMPLE 5

### Coupling Agents

The coupling agent is an organophosphorous derivative, it may be selected from phosphonic acids, monoesters and diesters of phosphonic acid or derivatives thereof, phosphoric acids, monoesters and diesters of phosphoric acid or derivatives thereof.

Phosphonic acids may be cited of generic formula:

Y-E-PO(OX)₂

with: X═H; R(R=Me, and, Na, iPr, tBu, L¹, NH⁴⁺, K, etc.); E=alkylene (preferably C2 to C60), alkenyl (preferably C2 to C60) or alkynyl radical (preferably C2 to C60), aryl radical and Y=known organic functions, biomolecules, colouring, luminescent, thermo-sensitive, pH-dependent functions, etc.

Coupling agents tested:
Octylphosphonic acid

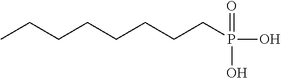

Dodecylphosphonic acid

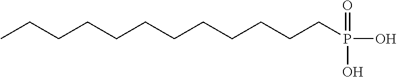 [SMILES: CCCCCCCCCCCCP(=O)(O)O]

Octadecylphosphonic acid

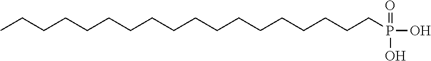 [SMILES: CCCCCCCCCCCCCCCCCCP(=O)(O)O]

Sodium diphosphonate

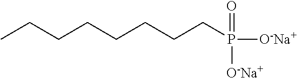

During this method, the organophosphorous groups are bound to the surface of the substrate via P—O-M bonds in which M represents a metal element of the substrate. These P—O-M bonds stem from the condensation of P—OX groups and/or the condensation of phosphoryl P═O groups with the surface hydroxyls. 3 OH sites would be accessible per nm² of inorganic substrate, the fixation could also be bi or tridentate. Thus, the organophosphorous derivatives can be fixed onto very numerous metal oxides such as iron oxide, titanium dioxide, chromium oxide, etc.

Only M-O—P bonds are brought into play during graftings on the substrate. These bonds are more stable than M-O—Si bonds created with an organosilane as coupling agent (state of the art: prior art search). This thus results in better stability of the grafting over time and faced with external agents when the coupling agent employed is an organophosphorous derivative. Moreover, phosphonic acids are very stable in water; consequently, it is not necessary to control the quantity of water at the surface of the metal oxide. Thus graftings may be implemented in water.

The subject matter of the invention is also a modified inorganic substrate obtained by the method. The substrate has a surface to which are bound organophosphorous groups able to have one or more organic substituents at the origin of the properties provided to the substrate.

For example, the field of cosmetics employs a large number of inorganic pigments. For better stability of cosmetics, this sector aims to confer on these pigments a hydrophobic character. The carbon chain of phosphonic acids of formula R—PO(OH)₂ confers the sought after hydrophobic character.

The hydrophobic character, in other words the efficiency of the grafting, has been qualified by means of hydrophoby and oil dispersion tests. The pigments used in cosmetics which require surface functionalisation in order to render them hydrophobic are of polar nature. Once functionalised, these become apolar and thus have an affinity with fatty bodies (also apolar). Conversely, there exists repulsion with polar binders such as water.

The hydrophoby test makes it possible to study the impregnation of porous materials by water when they are placed in contact. Thus, when, in a 250 ml beaker, 0.1 g of functionalised substrate is deposited on the surface of around 100 ml of osmosis purified water at 40° C. and after having left the whole to rest in an oven for 1 h at 40° C., the passage or not of the functionalised substrate into the aqueous phase reflects its hydrophobic character.

The oil dispersion test makes it possible to indicate the oil adsorption performances in cosmetic products. Thus, the reduction of the quantity of oil incorporated to obtain the complete wetting of the functionalised substrate compared to that necessary for a non-treated substrate reveals its hydrophobic character. To do so, 2 g of substrate, deposited beforehand on a watch glass, are mixed with a spatula for 3 to 4 minutes with oil deposited drop by drop using a burette.

UV-visible diffuse reflectance spectrophotometry then makes it possible to verify that the grafting method has not intrinsically altered the colour of the pigment.

Another example of interesting properties for the cosmetics sector is the hydrophilic character of the pigments. The studies carried out show that the pigments treated with a coupling agent such as (2-{2-[2-(2-hydroxy-ethoxy)-ethoxy]-ethoxy}-ethyl) phosphonic acid have a hydrophilic character.

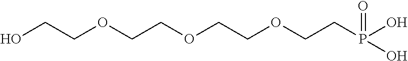 [SMILES: O=P(O)(O)CCOCCOCCOCCO]

Additional studies have demonstrated that other coupling agents such as carboxylic acids, sulphur containing derivatives could be employed during this method.
carboxylic acids R—COOH:

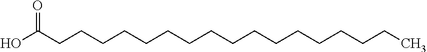 [SMILES: CCCCCCCCCCCCCCCCCC(=O)O]

Stearic acid
Thiols R—SH:

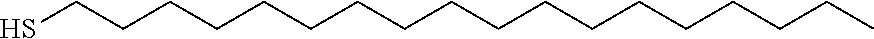 [SMILES: CCCCCCCCCCCCCCCCCCS]

Octadecanethiol
Quaternary ammonium salts:

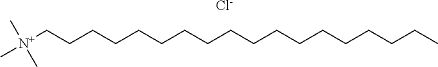 [SMILES: [Cl-] ; CCCCCCCCCCCCCCCCCC[N+](C)(C)C]

Octadecylammonium (Octadecyl Trimethyl Ammonium Chloride)

### EXAMPLE 6

### Example of Embodiment

Grafting with phosphonic acids bearing a single alkyl chain of 18 carbons (C18).

A) Inorganic Pigments

TABLE 5

| | Reaction | | Concentration |
|---|---|---|---|
| Pigment | time | Solvent | (mol · l⁻¹) |
| Black Iron Oxide | 5 min | 100% EtOH | 10⁻³ |
| | 5 min | 25% H₂O, 75% EtOH | 10⁻³ |
| Red Iron Oxide | 5 min | 100% EtOH | 10⁻³ |
| | 5 min | 25% H₂O, 75% EtOH | 10⁻³ |
| Yellow Iron Oxide | 5 min | 100% EtOH | 3 · 10⁻³ |
| | 5 min | 25% H₂O75% EtOH | 10⁻³ |
| Ultramarine | 5 min | 100% EtOH | 5 · 10⁻³ |
| | 5 min | 25% H₂O 75% EtOH | 5 · 10⁻³ |
| Titanium Dioxide | 5 min | 100% EtOH | 10⁻² |
| | 5 min | 25% H₂O, 75% EtOH | 5 · 10⁻³ |
| Chromium Oxide | 5 min | 100% EtOH | 5 · 10⁻³ |
| | 15 min | 25% H₂O, 75% EtOH | 10⁻² |
| Zinc Oxide | 5 min | 100% EtOH | 10⁻³ |
| | 5 min | 25% H₂O, 75% EtOH | 10⁻³ |
| Iron Blue | 5 min | 100% EtOH | 5 · 10⁻² |
| | 30 min | 10% H₂O 90% EtOH | 5 · 10⁻² |
| Chromium Oxide | 4 h | 100% EtOH | 10⁻² |
| Hydrated | | | |

*EtOH for ethanol

B) Composite Pigments

TABLE 6

| | Reac- | | Concen- |
|---|---|---|---|
| | tion | | tration |
| Composite pigment | time | Solvent | (mol · l⁻¹) |
| Mica (59 to 69%) | 5 min | 100% EtOH | 10⁻³ |
| TiO₂ (18 to 28%) | 5 min | 25% H₂O, 75% EtOH | 10⁻³ |
| Fe₃O₄ (1 to 6%) | | | |
| FeO(OH) (1 to 2%) | | | |
| Red7Calcium (8 to 12%) | | | |
| Mica (56 to 66%) | 5 min | 100% EtOH | 10⁻³ |
| TiO₂ (10 to 20%) | 5 min | 25% H₂O, 75% EtOH | 10⁻³ |
| Fe₃O₄ (2 to 12%) | | | |
| FeO(OH) (1 to 11%) | | | |
| Red7Calcium (8 to 12%) | | | |
| Fluorphlogopite (18 to 28%) | 5 min | 100% EtOH | 10⁻³ |
| TiO₂ (54 to 64%) | 5 min | 25% H₂O, 75% EtOH | 10⁻³ |
| Red40Aluminium (13 to 23%) | | | |
| Fluorphlogopite (23 to 33%) | 5 min | 100% EtOH | 5 · 10⁻² |
| TiO₂ (57 to 67%) | | | |
| Red7Calcium (5 to 15%) | | | |
| Mica (48 to 58%) | 5 min | 100% EtOH | 10⁻³ |
| TiO₂(32 to 42%) | 5 min | 25% H₂O, 75% EtOH | 10⁻³ |
| Red7Calcium (8 to 12%) | | | |
| Mica (46 to 56%) | 5 min | 100% EtOH | 10⁻³ |
| TiO₂ (34 to 44%) | 5 min | 25% H₂O, 75% EtOH | 10⁻³ |
| Red7Calcium (8 to 12%) | | | |
| Mica (50 to 60%) | 5 min | 100% EtOH | 10⁻³ |
| TiO₂ (29 to 39%) | 5 min | 25% H₂O, 75% EtOH | 10⁻³ |
| Red7Calcium (10 to 14%) | | | |
| Mica (49 to 59%) | 5 min | 100% EtOH | 10⁻³ |
| TiO₂ (36 to 46%) | 5 min | 25% H₂O, 75% EtOH | 10⁻² |
| Red7Calcium (3 to 7%) | | | |
| Mica (46 to 56%) | 5 min | 100% EtOH | 10⁻³ |
| TiO₂ (34 to 44%) | 5 min | 25% H₂O, 75% EtOH | 10⁻³ |
| Red28Aluminium (5 to 15%) | | | |
| Mica (44 to 54%) | 5 min | 100% EtOH | 10⁻³ |
| TiO₂ (32 to 42%) | 5 min | 25% H₂O, 75% EtOH | 10⁻³ |
| Blue1Aluminium (9 to 19%) | | | |
| Mica (48 to 58%) | 5 min | 100% EtOH | 10⁻³ |
| TiO₂ (28 to 38%) | 5 min | 25% H₂O, 75% EtOH | 10⁻³ |
| Yellow5Aluminium (9 to | | | |
| 19%) | | | |
| Mica (48 to 58%) | 5 min | 100% EtOH | 5 · 10⁻³ |
| TiO₂ (28 to 38%) | 5 min | 25% H₂O, 75% EtOH | 5 · 10⁻³ |
| Red6Sodium (9 to 19%) | | | |
| Mica (47 to 57%) | 5 min | 100% EtOH | 10⁻³ |
| TiO₂ (31 to 41%) | 5 min | 25% H₂O, 75% EtOH | 10⁻³ |
| Red30Aluminium (7 to 17%) | | | |

TABLE 7

| | Reac- | | Concen- |
|---|---|---|---|
| | tion | | tration |
| Composite pigment | time | Solvent | (mol · l⁻¹) |
| Mica (43 to 54%) | 5 min | 100% EtOH | 10⁻² |
| TiO₂ (46 to 57%) | 5 min | 25% H₂O, 75% EtOH | 10⁻² |
| Mica (55 to 65%) | 5 min | 100% EtOH | 10⁻² |
| TiO₂ (45 to 55%) | 5 min | 25% H₂O, 75% EtOH | 10⁻² |
| Mica (62 to 73%) | 5 min | 100% EtOH | 10⁻² |
| TiO₂ (27 to 38%) | 5 min | 25% H₂O, 75% EtOH | 10⁻² |
| Mica (77 to 87%) | 5 min | 100% EtOH | 10⁻² |
| TiO₂ (13 to 23%) | 5 min | 25% H₂O, 75% EtOH | 10⁻² |
| Mica (64 to 74%) | 5 min | 100% EtOH | 10⁻² |
| TiO₂ (26 to 36%) | 5 min | 25% H₂O, 75% EtOH | 10⁻² |
| Mica (52 to 63%) | 5 min | 100% EtOH | 10⁻² |
| TiO₂ (37 to 48%) | 5 min | 25% H₂O, 75% EtOH | 10⁻² |
| Mica (47 to 58%) | 5 min | 100% EtOH | 10⁻² |
| TiO₂ (42 to 53%) | 5 min | 25% H₂O, 75% EtOH | 10⁻² |
| Mica (45 to 56%) | 5 min | 100% EtOH | 10⁻² |
| TiO₂ (44 to 55%) | 5 min | 25% H₂O, 75% EtOH | 10⁻² |
| Mica (43 to 54%) | 5 min | 100% EtOH | 10⁻² |
| TiO₂ (46 to 57%) | 5 min | 25% H₂O, 75% EtOH | 10⁻² |
| Mica (37 to 48%) | 5 min | 100% EtOH | 10⁻² |
| TiO₂ (52 to 63%) | 5 min | 25% H₂O, 75% EtOH | 10⁻² |
| Mica (54 to 64%) | 5 min | 100% EtOH | 10⁻² |
| Fe₂O₃ (36 to 46%) | 5 min | 25% H₂O, 75% EtOH | 10⁻² |
| Mica (52 to 62%) | 5 min | 100% EtOH | 10⁻² |
| Fe₂O₃ (38 to 48%) | 5 min | 25% H₂O, 75% EtOH | 10⁻² |
| Mica (62 to 72%) | 5 min | 100% EtOH | 10⁻² |
| Fe₂O₃ (28 to 38%) | 5 min | 25% H₂O, 75% EtOH | 10⁻² |
| Mica (55 to 70%) | 5 min | 100% EtOH | 10⁻² |
| TiO₂ (28 to 38%) | 5 min | 25% H₂O, 75% EtOH | 10⁻² |
| Fe₂O₃ (2 to 7%) | | | |

C) Excipents

TABLE 8

| | Reaction | | Concentration |
|---|---|---|---|
| Excipient | time | Solvent | (mol · l⁻¹) |
| Mica | 24 H | 100% EtOH | 10⁻² |
| | 24 H | 25% H₂O, 75% EtOH | 10⁻² |
| Fluorphlogopite | 24 H | 100% EtOH | 10⁻² |
| | 24 H | 25% H₂O, 75% EtOH | 10⁻² |
| Talc | 5 min | 100% EtOH | 10⁻² |
| | 5 min | 25% H₂O, 75% EtOH | 10⁻² |

### EXAMPLE 7

### Organic Pigments

Organic pigments are mainly lakes.
True pigments are pigments that precipitate while forming an insoluble organic structure.
Inks are obtained by precipitation of a soluble colorant in water as metal salts.
Lakes are obtained by absorption of a soluble colorant in water in an insoluble inorganic substrate.

### Examples:

TABLE 9

| | | | | | % |
|---|---|---|---|---|---|
| | | Organic | % | Inorganic | Inorganic |
| Reference | Name | molecule | Pigment | substrate | substrate |
| C69-002 C69-4424 C69-4537 | SunCROMA ™ FD&C Yellow 5 Al. | 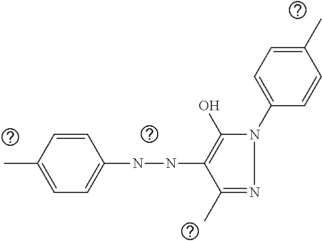 | 15 24 38 | Al(OH)₃ Al(OH)₃ Al(OH)₃ | 82.5 73.5 59.5 |
| C70-5270 | SunCROMA ™ FD&C Yellow 6 Al. | 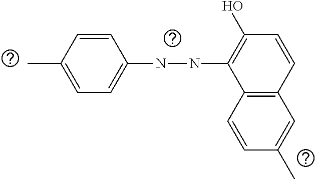 | 40 | Al(OH)₃ | 57. 5 |
| C19-003 C19-011 C19-012 C19-025 | SunCROMA ™ D&C Red 7 Ca. SunCROMA ™ D&C Red 7 Ca. SunCROMA ™ D&C Red 6 Ba. SunCROMA ™ D&C Red 7 Ca. | 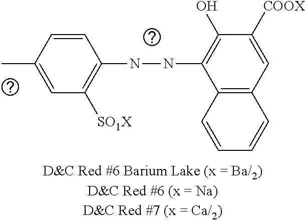 | 60 38 42 47 | BaSO₄ BaSO₄ + resin BaSO₄ + resin BaSO₄ + calcium resin | 30 52 48 43 |
| C24-012 | SunCROMA ™ D&C Red 34 Ca. | 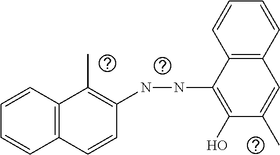 [SMILES: Cc1cc2ccccc2c(NNc2ccc3ccccc3c2C)c1O] | 83 | Resin | 13.8 |
| | | | | | % |
| | | Organic | % | Inorganic | Inorganic |
| Reference | Lake | molecule | Pigment | substrate | substrate |
| C14-023 C14-6602 C14-6623 | SunCROMA ™ D&C Red 27 Al. SunCROMA ™ Red D&C 27 Al. SunCROMA ™ D&C Red 28 Al. | 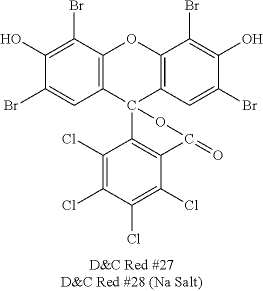 [SMILES: O=C1OC2(c3cc(Br)c(O)c(Br)c3Oc3c2cc(Br)c(O)c3Br)c2c(Cl)c(Cl)c(Cl)c(Cl)c21] | 24 20 24 | Al(OH)₃ + Aluminium benzoate Al(OH)₃ + Aluminium benzoate Al(OH)₃ + Aluminium benzoate | 72.3 76.3 72.3 |
| C14-6634 C14-032 | SunCROMA ™ D&C Red 22 Al. D&C Red 21 | 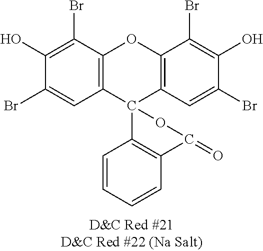 [SMILES: O=C1OC2(c3ccccc31)c1cc(Br)c(O)c(Br)c1Oc1c2cc(Br)c(O)c1Br] | | | |
| | | | | | % |
| | | Organic | % | Inorganic | Inorganic |
| Reference | Name | molecule | Pigment | substrate | substrate |
| C39-4434 | SunCROMA ™ FD&C Blue 1 Al. | 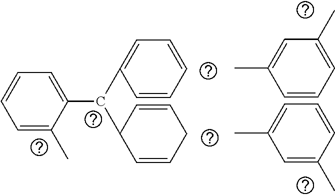 | 12 | Al(OH)₃ | 85.5 |

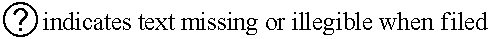

### EXAMPLE 8

### Method Known as by “Dry Means”

The principle of this method is grafting by placing in contact the coupling agent and pigments (substrates). The pigments thus treated may be organic or inorganic (or composites). This method also makes it possible to treat excipients and effect pigments.

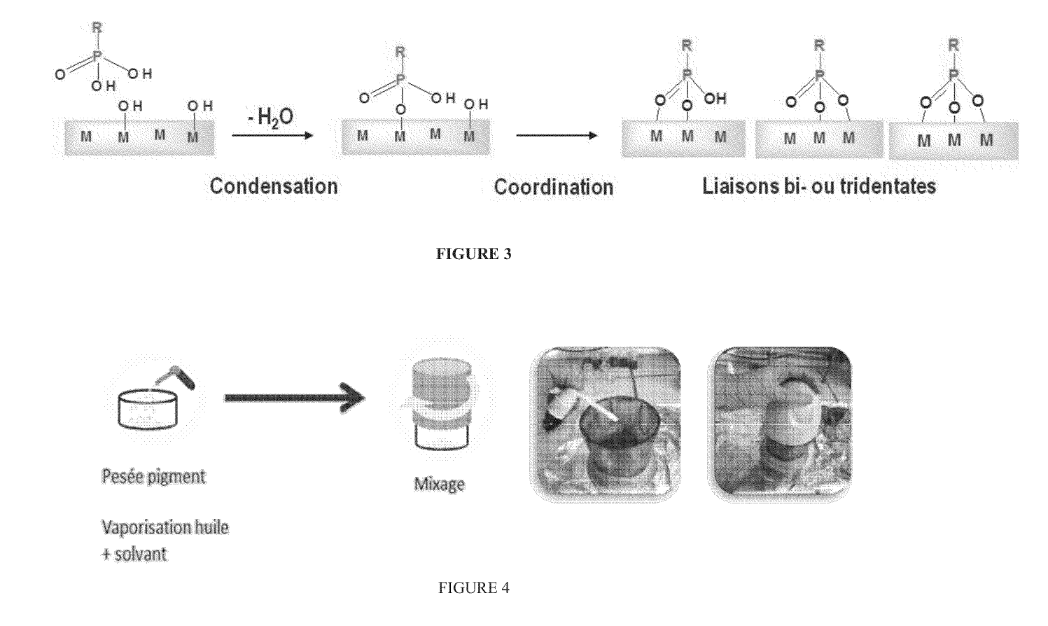

The method takes place in three steps (See FIG. 4: Diagram of the method by dry means with photographs of grafting by dry means):

1 Vaporisation of the mixture containing the coupling agent and the suitable solvent on the surface of the pigment (substrate) to be treated.
2. Homogeneous placing in contact of the solvent/coupling agent mixture and the pigment to be treated (substrate), particularly by means of a mixer or stirrer.

### 3. Drying.

### Step 1:

The solution is prepared by mixing a coupling agent with a selected solvent. The emulsion is then entirely vaporised on the pigments to be treated.

The weight percent of the coupling agent in solution varies as a function of the weight ratio of the couple (coupling agent/pigment to be treated). It is generally considered as sufficient from 3%. The choice of the solvent (H₂O, ethanol (EtOH) or H₂O/EtOH mixture) also varies as a function of the coupling agent/pigment couple. Conventional organic solvents can also be employed but current environmental considerations tend to see their uses reduced.

### Step 2:

The placing in contact of the entire surface of the pigments with the coupling agent takes place using an internal mixer in order that said placing in contact is homogeneous. The internal mixer is equipped with a thermoregulator which makes it possible to regulate the temperature. The final solvent/coupling agent+pigment mixture thus obtained is in the form of a powder.

### Step 3:

The final mixture is heated to a temperature comprised between 40° C. and 80° C.

Example of embodiment:

Weighing out 5 g of pigment in a mixer

Weighing out 0.5 g of solvent in a sprayer

Adding the coupling agent to the sprayer:

0.5 g for 10% grafting by weight, 0.25 g for 5% and 0.15 g for 3%

Vaporising the solution on the powder

Mixing for 1 min (with a mixer)

Drying at 80° C. for 12 hours.

1. Method of surface modification of a pigment or of a composition comprising at least a pigment, an excipient and, if need be, a lake, said pigment being a metal oxide, a metal complex or a derivative thereof, characterised in that it comprises the following steps:
A) placing in contact said pigment or said composition comprising at least a pigment with a coupling reagent in a solvent and in conditions making it possible to form a chemical bond, preferably covalent or iono-covalent, between said pigment and said coupling reagent; and
B) recovering said pigment or said composition comprising at least a pigment, the surface of which has thus been modified.

2. Method of surface modification according to claim 1, characterised in that said pigment or said composition comprising at least a pigment is intended to be used in cosmetics, in inks or in paint, preferably in cosmetics.

3. Method of surface modification according to claim 1 or 2, characterised in that said coupling reagent further comprises a function that does not react with the surface of said pigment, but which provides the desired physical-chemical property.

4. Method of surface modification according to one of claims 1 to 3, characterised in that said pigment is a particle, porous or not, which can exist in different geometric shapes (sphere, rod, wire, tube) the size of which is comprised between 500 μm and 1 nm, preferably between 50 μm and 50 nm.

5. Method of surface modification according to one of claims 1 to 4, characterised in that said pigment is selected from groups of pigments consisting of:
1) Inorganic pigments; and
2) Composite pigments.

6. Method of surface modification according to one of claims 1 to 5, characterised in that said excipient is selected from excipients of natural or synthetic origin, preferably selected from the following group:
Mica of formula X₂Y₄₋₆Z₈O₂₀(OH,F)₄ with X═K, Na, Ca, Ba, Rb, Cs; Y═Al, Mg, Mn, Cr, Ti, Li etc. and Z═Si, Al, Fe³⁺, Ti;
Synthetic fluorphlogopite of formula KMg₃(AlSI₃O₁₀)F₂;
Talc of formula Mg₃Si₄O₁₀(OH)₂,
Sericite of formula KAl₂ [(OH,F)₂|AlSi₃O₁₀]/KAl₂ [(OH,F)₂|AlSi₃O₁₀];
Silica of formula SiO₂, and
Alumina of formula Al₂O₃.

7. Method of surface modification according to one of claims 1 to 6, characterised in that said pigment is an inorganic pigment being able to be composed of the following different chemical elements: F, Na, Mg, Al, Si, P, S, K, Ca, Ti, V, Cr, Mn, Fe, Co, Ni, Cu, Zn, Se, Zr, Mo, Ru, Cd, Sn, Sb, or W.

8. Method of surface modification according to one of claims 1 to 7, characterised in that said pigment is a composite pigment formed of one or more inorganic pigments associated with one or more excipients in the presence or not of a lake.

9. Method of surface modification according to one of claims 1 to 8, characterised in that said pigment is an inorganic pigment selected from the group constituted of the following pigments, Black Iron Oxide, Red Iron Oxide, Yellow Iron Oxide, Manganese Violet, Ultramarine Blue, Titanium Dioxide, Chromium Oxide Green, Zinc Oxide, Iron Blue, Chromium Oxide Hydrated.

10. Method of surface modification according to one of claims 1 to 8, characterised in that said pigment is a composite pigment comprising an inorganic pigment, an excipient and, if need be, a lake selected from the group constituted of the following composite pigments:
Mica (59 to 69%), TiO₂ (18 to 28%), Fe₃O₄ (1 to 6%), FeO(OH) (1 to 2%) and Red7Calcium (8 to 12%);
Mica (56 to 66%), TiO₂ (10 to 20%), Fe₃O₄ (2 to 12%), FeO(OH) (1 to 11%) and Red7Calcium (8 to 12%);
Synthetic fluorphlogopite (18 to 28%), TiO₂ (54 to 64%) and Red40Aluminium (13 to 23%);
Synthetic fluorphlogopite (23 to 33%), TiO₂ (57 to 67%) and Red7Calcium (5 to 15%);
Mica (48 to 58%), TiO₂ (32 to 42%) and Red7Calcium (8 to 12%);
Mica (46 to 56%), TiO₂ (34 to 44%) and Red7Calcium (8 to 12%);
Mica (50 to 60%), TiO₂ (29 to 39%) and Red7Calcium (10 to 14%);
Mica (49 to 59%), TiO₂ (36 to 46%) and Red7Calcium (3 to 7%);
Mica (46 to 56%), TiO₂ (34 to 44%) and Red28Aluminium (5 to 15%);
Mica (44 to 54%), TiO₂ (32 to 42%) and Blue1Aluminium (9 to 19%);
Mica (48 to 58%), TiO₂ (28 to 38%) and Yellow5Aluminium (9 to 19%);
Mica (48 to 58%), TiO₂ (28 to 38%) and Red6Sodium (9 to 19%);
Mica (47 to 57%), TiO₂ (31 to 41%) and Red30Aluminium (7 to 17%);
Mica (43 to 54%) and TiO₂ (46 to 57%);
Mica (55 to 65%) and TiO₂ (45 to 55%);
Mica (62 to 73%) and TiO₂ (27 to 38%);
Mica (77 to 87%) and TiO₂ (13 to 23%);
Mica (64 to 74%) and TiO₂ (26 to 36%);
Mica (52 to 63%) and TiO₂ (37 to 48%);
Mica (47 to 58%) and TiO₂ (42 to 53%);
Mica (45 to 56%) and TiO₂ (44 to 55%);
Mica (43 to 54%) and TiO₂ (46 to 57%);
Mica (37 to 48%) and TiO₂ (52 to 63%);
Mica (54 to 64%) and Fe₂O₃ (36 to 46%);
Mica (52 to 62% and Fe₂O₃ (38 to 48%);
Mica (62 to 72%) and Fe₂O₃ (28 to 38%); and
Mica (55 to 70%), TiO₂ (28 to 38%) and Fe₂O₃ (2 to 7%).

11. Method of surface modification according to one of claims 1 to 10, characterised in that said solvent is selected from “eco-friendly” (non-toxic) solvents, preferentially cosmetically acceptable, even more preferably selected from the group constituted of the following solvents: ethanol, water, ethanol/water mixture in proportions ranging from 100%/0% to 0%/100%.

12. Method of surface modification according to one of claims 1 to 10, characterised in that said solvent is selected from organic solvents, preferably selected from the group constituted of the following organic solvents: tetrahydrofuran, dimethylsulphoxide, dimethylformamide, cyclohexane, pentane, acetone, toluene, dichloromethane and isopropanol.

13. Method of surface modification according to one of claims 1 to 12, characterised in that said coupling reagent is selected from the group constituted of coupling reagents of generic formula:
Y-E-Z (I)
in which:
Y is a known organic function, a colorant, luminescent, pH-dependent, thermo-sensitive chemical group or a biomolecule;
E is a radical selected from alkylene, alkenyl, alkynyl or aryl radicals, preferably from C2 to C60 for alkylene, alkenyl, alkynyl radicals, or selected from poly(oxyethylene) radicals comprising a number of oxyethylene units comprised between 4 and 500;
Z is a radical selected from: —PO(OX)₂ (with X═H, Me, and, iPr, tBu, Na, Li, NH4⁺, K), —O—PO(OX)₂ (with X═H, Me, and, iPr, tBu, Na, Li, NH4⁺, K), —COOH, —SH, —SO₃H, quaternary ammonium salts.

14. Method of surface modification according to one of claims 1 to 13, characterised in that said coupling reagent is selected from the group constituted of the following coupling reagents:
phosphonic acids and derivatives thereof such as octylphosphonic acid, dodecylphosphonic acid, octadecylphosphonic acid, (2-{2-[2-(2-hydroxy-ethoxy)-ethoxy]-ethoxy}-ethyl) phosphonic acid, sodium diphosphonate;
carboxylic acids and derivatives thereof such as octanoic acid, dodecanoic acid, stearic acid, oleic acid, linoleic acid;
thiols and derivatives thereof such as octadecanethiol;
amine functions and derivatives thereof such as octadecylammonium.

15. Method of surface modification according to one of claims 1 to 14, characterised in that it comprises the following steps:
a) placing in contact said pigment or said composition comprising at least an inorganic pigment with a coupling reagent in a solvent and in conditions making it possible to form a chemical bond, preferably covalent or iono-covalent, between said pigment and said coupling reagent;
b) eliminating the coupling reagent not having reacted with said pigment;
c) if need be, washing the pigment thus modified;
d) if need be, drying the pigment thus modified on its surface obtained at step b) or c) or the pigment composition; and
e) recovering said pigment or said composition comprising at least an inorganic pigment, the surface of which has thus been modified.

16. Method of surface modification according to one of claims 1 to 14, characterised in that it comprises the following steps:
a) placing in contact said pigment or said composition comprising at least an inorganic pigment with a coupling reagent in a solvent and in conditions making it possible to form a covalent or iono-covalent bond between said pigment and said coupling reagent;
b) eliminating the coupling reagent not having reacted with said pigment;
c) if need be, washing the pigment thus modified;
d) drying the pigment thus modified at its surface obtained at step b) or c) or the pigment composition; and
e) if need be, recovering said pigment or said composition comprising at least an inorganic pigment, the surface of which has thus been modified, in the form of a powder.

17. Method of surface modification according to one of claim 15 or 16, characterised in that the steps of washing (and/or rinsing) and recovering the pigment, the surface of which has been modified, are carried out in the presence of solvent by mechanical separation (decantation, centrifugation), filtration in depth or on support preferably by filtration on membrane support.

18. Method of surface modification according to one of claims 15 to 17, characterised in that the drying step is carried out by heating, preferably at a temperature comprised between 20° C. and 150° C.

19. Method of surface modification according to one of claims 14 to 18, characterised in that the step of washing and/or rinsing is followed by a step in which the pigment or the composition of pigments obtained is subjected to ultrasounds.

20. Method of preparing a pigment powder or a composition comprising at least a pigment, preferably intended to be used in cosmetics, in paint or in inks, preferably in cosmetics, characterised in that it comprises the following steps:
1) modifying the surface of the pigment by a method according to one of claims 15 to 19; and
2) a step of recovering said pigment or said composition comprising at least a pigment, the surface of which has thus been modified obtained at step 1), in the form of a powder.

21. Method of preparing a pigment powder or a dry composition comprising at least a pigment, said pigment being selected from inorganic, composite or organic pigments, preferably said powder or dry composition being intended to be used in cosmetics, in paint or in inks, preferably in cosmetics, characterised in that it comprises the following steps:
1) modifying the surface of the pigment by a method according to one of claims 1 to 15, in which method a pigment or a composition comprising at least a pigment is placed in contact with a reagent or coupling agent in a solvent and in conditions making it possible to form a chemical bond, preferably covalent or iono-covalent, between said pigment and said coupling reagent, and characterised in that the solvent/coupling reagent mixture represents by weight less than 50% of the weight of pigment, preferably comprised between 3% and 10%;
2) after mixing, a step of drying, preferably by heating, the mixture obtained at step 1) so as to obtain a pigment powder or a dry composition, preferably by heating to a temperature comprised between 20° C. and 80° C.

22. Method of preparing a pigment powder or a dry composition according to claim 21, characterised in that at step 1, the placing in contact of the coupling agent/solvent mixture with said pigment or composition is carried out by vaporisation of the solvent/coupling agent mixture on the surface of the pigment.

23. Pigment powder capable of being obtained by a method according to one of claims 15 to 22, coated with a monolayer of coupling agent in which the free terminal functions (Y) confer to the pigments the desired physical-chemical properties.

24. Powder of dispersed pigments capable of being obtained by a method according to one of claims 15 to 23, preferably intended to be used in cosmetics, and in which method the surface modification is intended to provide properties of dispersion, stability (time, temperature, radiation), texturing but can also make it possible to simplify the formulation, to protect the pigment or to limit the use of toxic products.

25. Pigment or pigment composition capable of being obtained by a method according to one of claims 1 to 24, intended to be used in cosmetics, in paint or in inks, preferably in cosmetics, characterised in that it is contained in an eye shadow, a blush, a lipstick, a cream, a gel, a soap bar or any form of cosmetic product.

26. Pigment or pigment composition capable of being obtained by a method according to one of claims 1 to 20, characterised in that the method implements the following elements:
1)—Coupling reagent: Phosphonic acid (C18);
Pigments:
Inorganic pigments selected from: Black Iron Oxide; Red Iron Oxide; Yellow Iron Oxide; Titanium Dioxide; Chromium Oxide Green; Ultramarine Blue; Zinc Oxide; Iron Blue; or Chromium Oxide Hydrated;
Composite pigments;
Excipients: Mica; Fluorphlogopite; or Talc,
or
2)—Coupling reagent: Carboxylic acid (C18)
Pigments:
Inorganic pigments selected from: Black Iron Oxide; Red Iron Oxide; Yellow Iron Oxide; Titanium Dioxide; Chromium Oxide Green; Zinc Oxide; or Chromium Oxide Hydrated;
Composite pigments selected from: Mica (59 to 69%), TiO₂ (18 to 28%), Fe₃O₄ (1 to 6%), FeO(OH) (1 to 2%) and Red7Calcium (8 to 12%); Mica (56 to 66%), TiO₂ (10 to 20%), Fe₃O₄ (2 to 12%), FeO(OH) (1 to 11%) and Red7Calcium (8 to 12%); or Mica (46 to 56%), TiO₂ (34 to 44%) and Red28Aluminium (5 to 15%);
Excipient: Talc,
or
3)—Coupling reagent: Thiol (C18)
Pigments:
Inorganic pigments selected from: Black Iron Oxide; Red Iron Oxide; Yellow Iron Oxide; Titanium Dioxide; Chromium Oxide Green; Zinc Oxide; or Iron Blue;
Composite pigments selected from: Mica (59 to 69%), TiO₂ (18 to 28%), Fe₃O₄ (1 to 6%), FeO(OH) (1 to 2%) and Red7Calcium (8 to 12%); or Mica (56 to 66%), TiO₂ (10 to 20%), Fe₃O₄ (2 to 12%), FeO(OH) (1 to 11%) and Red7Calcium (8 to 12%),
or
4)—Coupling reagent: Ammonium salts (C18)
Pigments:
Inorganic pigments selected from: Manganese Violet; or Zinc Oxide;
Composite pigments;
Excipients: Mica; Fluorphlogopite; Talc, Silica, Sericite or Alumina,
or
5)—Coupling reagent: (2-{2-[2-(2-hydroxy-ethoxy)-ethoxy]-ethoxy}-ethyl) phosphonic acid
Pigments:
Inorganic pigments selected from: Black Iron Oxide; Red Iron Oxide; Yellow Iron Oxide; Ultramarine; or Zinc Oxide
Composite pigments: Mica (59 to 69%) TiO₂ (18 to 28%) Fe₃O₄ (1 to 6%) FeO(OH) (1 to 2%) Red7Calcium (8 to 12%);

27. Eye shadow formulation of following composition:
Mica, Lauroyl Lysine 48% by weight;
Pigments modified according to claim 25 or 26: 40% by weight;
Petrolatum 2% by weight;
Dimethicone 5% by weight; and
Caprilic/Capric Triglyceride 5% by weight.

28. Foundation formulation of the following composition:
Stearoyl inulin 2% by weight;
Isotridecyl isononanate 5% by weight;
Dimethicone copolyol 1.7% by weight;
Cyclomethicone 13.4% by weight;
Pigments modified according to claim 25 or 26: 6.9% by weight;
Sodium chloride 1% by weight;
Methylparaben 0.2% by weight; and
Purified water qs.
